(19) United States
(12) Patent Application Publication
(10) Pub. No.: US 2014/0213901 A1
(43) Pub. Date: Jul. 31, 2014

(54) System & Method for Delineation and Quantification of Fluid Accumulation in EFAST Trauma Ultrasound Images

(71) Applicant: Karen Shackelford, Jackson, MS (US)

(72) Inventor: Karen Shackelford, Jackson, MS (US)

(21) Appl. No.: 14/167,448

(22) Filed: Jan. 29, 2014

Related U.S. Application Data
(60) Provisional application No. 61/757,886, filed on Jan. 29, 2013.

(51) Int. Cl. A61B 8/08

(52) U.S. Cl. CPC A61B 8/5207
USPC 600/437

(57) ABSTRACT

eFAST ultrasound is a cost effective assessment procedure using software to enable detection of free fluid/air accumulation in trauma cases using images from portable ultrasound study.

### STATEMENT REGARDING PRIORITY

This application is a continuation of, and claims the benefit of the priority date of, provisional application Ser. No. 61/757,886 filed on Jan. 29, 2013. The present application incorporates 61/757,886 by reference in its entirety.

### FIELD

The disclosure pertains to the field of extended Focused Assessment with Sonography for Trauma (FAST U/S).

### BACKGROUND

Ultrasound refers to a spectrum of high frequency sound which is above the range of human hearing. In ultrasound imaging, when ultrasound waves hit the molecules of an object, some of the waves are reflected back and some are transmitted through the material. This occurs at a sound-sound interface, and this quality can be represented by an image.

The FAST ultrasound examination is an acronym for Focused Assessment with Sonography in Trauma. The term Focused Assessment with Sonography for Trauma (FAST) was coined by Rozycki et al in 1996 and has persisted as the accepted acronym for the trauma ultrasound evaluation. The basic four-view examination (perihepatic, perisplenic, pelvic, and pericardial views) has become the foundation of the FAST examination. In order to perform a FAST exam, an operator must be trained to place an ultrasound probe in the correct orientation in the sub-xiphoid space, the right upper quadrant, the left upper quadrant, and the suprapubic region. The probe placement is easy to learn, but the interpretation of resultant images is more difficult, and care must be taken as any misinterpretation can be disastrous for the patient.

An Extended FAST (eFAST) exam includes the element of ultrasound investigation of the pleural space in the thoracic cavity, which can highlight the accumulation of blood or air in the potential space between the visceral and parietal pleura. The rapid, non-invasive, and practical nature of ultrasound for bedside evaluation of critically injured patients has changed the evaluation of trauma. These examinations are performed using modern medical handhelds or conventional ultrasound machines which have the capability to record real-time high-resolution imaging without the use of ionizing radiation, and storage of images obtained in various formats (TIFF, AVI etc). These are portable images, easy to handle and transport.

Free fluid in the peritoneum or abdomen is an abnormal finding, and represents hemorrhage in the abdominal cavity. Likewise, fluid around the heart, in the pericardium, represents the abnormal finding of cardiac tamponade. On ultrasound, a dark stripe in Morrison's pouch (the space between the liver and the kidney), Pouch of Douglas (the vesicular-rectal space or between bladder and rectum in the pelvis), and perisplenic area (around spleen) represent unusual bleeding in trauma. The pericardial space is also routinely examined as part of the FAST examination, and if there is fluid in the pericardial space, it can be decompressed if it is causing hemodynamic instability or cardiogenic shock.

The delineated area of free fluid or air is called an anechoic area, as there is no reflection of the ultrasound wave emitted by an ultrasound probe, and these areas are shown in black or as voids on the ultrasound image. 200 cc.-300 cc. of fluid must generally accumulate to show up on ultrasound in the space between the liver and kidney. Ultrasound image analysis must isolate and delineate this area of image blackness representing free fluid, which is not currently quantified in ultrasound. The examination utilized in the FAST exam is based on a binary decision tree—yes, there is free fluid, or no, there is not. Serial exams can reveal increase in size of any anechoic area, and can be used to estimate the rate of hemorrhage.

Although serial examinations are performed to determine an estimated rate of hemorrhage, it is proposed that the fluid accumulated is first delineated and quantified using image processing techniques applied to the images obtained through the exam recommended by the American College of Surgeons, the FAST.

Further, eFAST allows the identification, through the same methodology described below, to delineate free air and/or fluid in the pleural spaces, representing pneumothorax or hemothorax.

When assessing ultrasound in the evaluation of the blunt trauma patient, calculations such as sensitivity and specificity may be misleading. Consequently, the question should be, How does this ultrasound result affect the patient for whom I am caring right now? In detecting intra-abdominal injuries in trauma patients, the governing paradigm always has been to recognize those patients who require laparotomy, and to determine disposition quickly to prevent further morbidity or mortality. Underlying this paradigm is the understanding that ongoing evaluation through serial examinations or other imaging may be needed to determine whether a patient has any intra-abdominal injury or intra-thoracic injury requiring an invasive procedure. Most experts would concur that ultrasound has performed best when limited to detecting free intraperitoneal fluid and air in the pleural space. Historically, the presence of any intra-peritoneal fluid in an unstable patient has indicated a significant intra-abdominal injury and warranted an immediate exploratory laparotomy. However, if the injury is not identified in the abdominal cavity, the decision tree changes. If a thoracic or orthopedic surgeon is instead required, the patient may be transferred to a higher level of trauma care.

The delineation of the presence of any intraperitoneal fluid and quantifying the volume of fluid shall enable the expert to arrive at the right clinical / surgical pathways.

### BRIEF DESCRIPTION OF THE DRAWINGS

FIG. 1 illustrates an example of a cloud resources based system for eFAST U/S;

FIG. 2 illustrates more details of the system shown in FIG. 1;

FIG. 3 illustrates one embodiment of a method for the delineation and quantification of fluid accumulation from eFAST ultrasound images;

FIG. 4 illustrates the invention in one embodiment comprising a set of image processing operations that are part of the delineation and quantification of fluid accumulation method; and

FIG. 5 illustrates examples of the image processing operations in FIG. 4. The object outlined have been enhanced by the methods described herein.

### DETAILED DESCRIPTION OF PREFERRED EMBODIMENTS

The needs of high reproducibility and increasing efficiency motivate the development of computer-assisted and automated segmentation. These automated procedures segment different regions in medical images by applying different types of image segmentation methods. An effective and correct diagnosis of ultrasound image is very important to avoid segmentation of normal fluids, cavities, tissues and organs, which can lead to misdiagnoses in evaluation and treatment of the patient. Thus, automated segmentation will be extremely useful to help clinicians make a diagnosis and decision pertinent to patient care. Current ultrasound images may be of poor quality, due to the relatively low resolution and reduced contrast of the images.

A significant disadvantage in the interpretation of ultrasound images is the poor quality of images, which are affected by speckle or other noise. Speckle noise is a granular noise due to random interference pattern in an image formed. Noise reduction in general could use anyone of different methods available. Anisotropic diffusion is one such process. Speckle reduction is a variant of anisotropic diffusion filter for ultrasound based images. Generally understood and MatLab implementations are available.

Applying one or more noise reduction methods such as speckle reducing anisotropic diffusion (SRAD) and contrast enhancement in the pre-processing phase make the image clearer before applying the morphology operations.

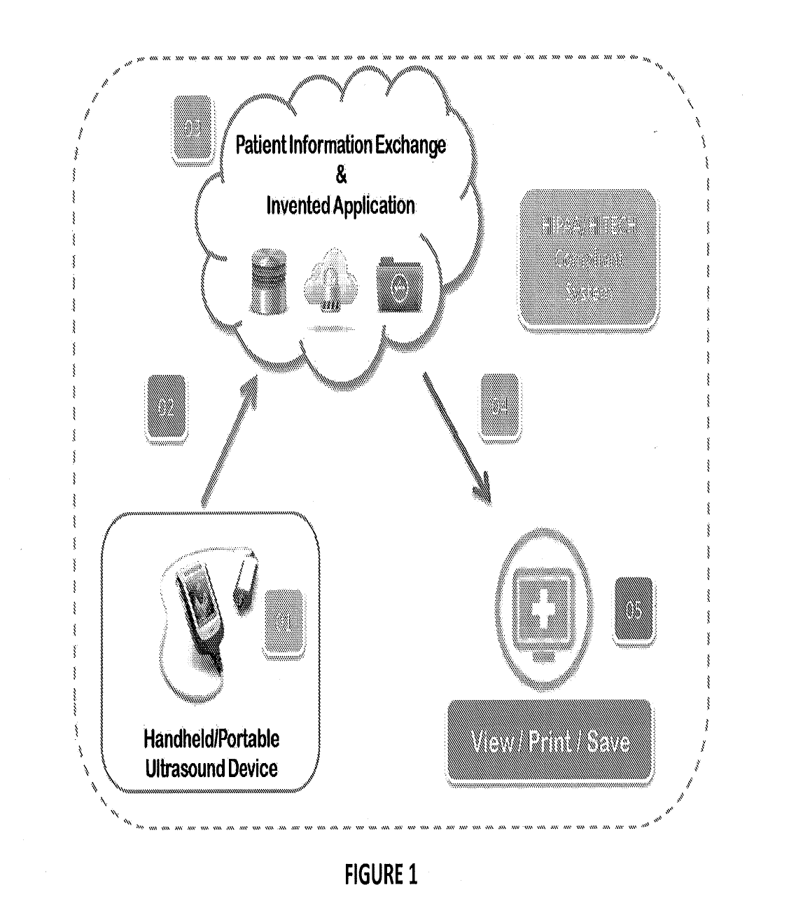

FIG. 1 illustrates one embodiment of a cloud resources-based system for eFAST U/S and

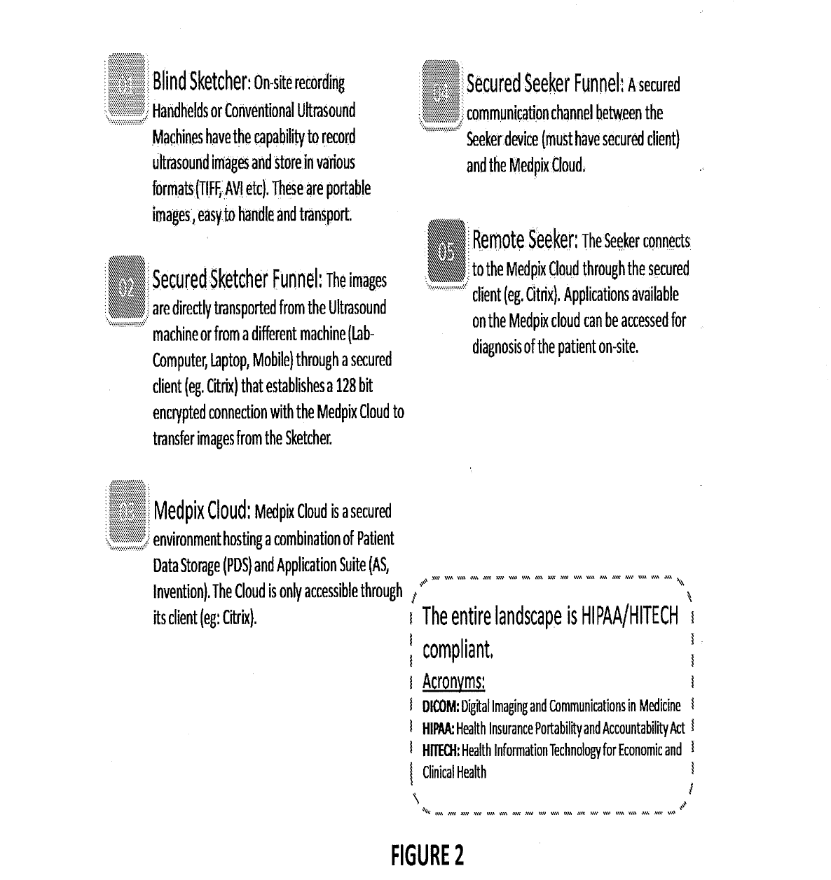

FIG. 2 illustrates more details of one embodiment of the system shown in FIG. 1. The system may be housed in the cloud (and utilize cloud computing resources) as indicated by label 3 in FIG. 1 that is described in more detail in FIG. 2. The other aspects shown in FIG. 1 (and the labels) are also described in more detail in FIG. 2.

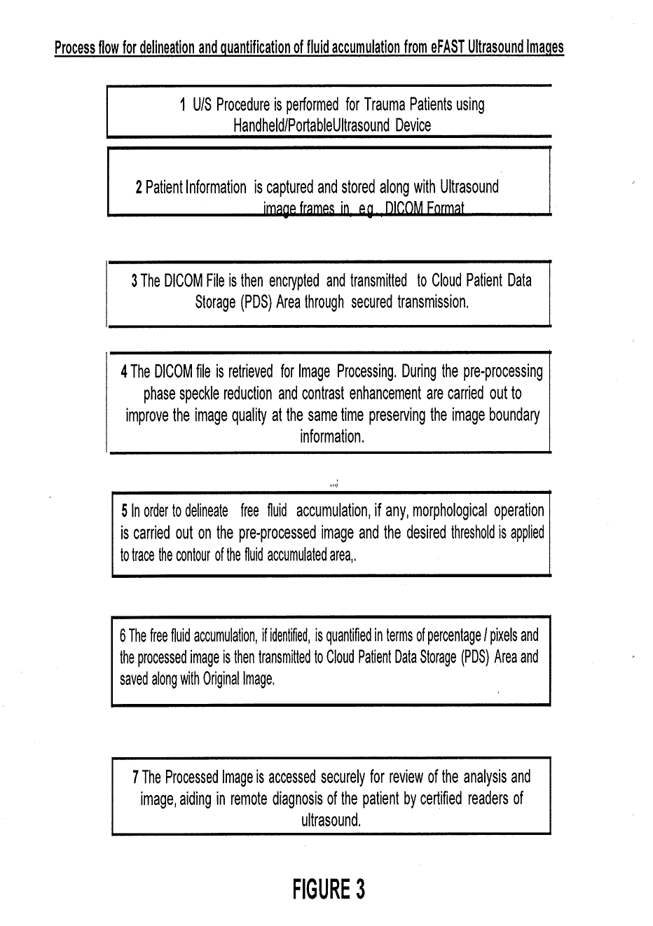

FIG. 3 illustrates one embodiment of a method for the delineation and quantification of fluid accumulation from eFAST ultrasound images that has the different processes shown in FIG. 3.

The currently used diagnostic peritoneal lavage is an invasive test involving an incision in the area distal to the umbilicus, requiring infusion of fluid, and siphoning off the mixture—the observed red blood cell count dictates whether the bleed requires surgery or observation. It is of clear benefit to the patient to have the presence of fluid in abnormal locations noted, and to have any fluid increase quantified as such, although the amount of fluid is not measured currently. It is of even greater benefit to determine the presence of fluid in abnormal locations without an invasive procedure, as is disclosed herein.

Currently, most doctors, nurse practitioners, and EMTs cannot analyze and act upon ultrasound images, even if they have enough experience to identify essentially what is going on. Ultrasound interpretation is outside of their scope of practice, and a trained and certified physician must look at the study to act confidently and efficiently on the results of the eFAST exam. Analysis is something a field practitioner can communicate easily on the phone prior to medical control access of the analysis and review of the image study. This makes preliminary decision making a very abbreviated process, especially in comparison to the modalities currently utilized in centers where ultrasound is not available in the emergency department.

As shown in FIG. 3, the first step 1 is to perform an ultrasound using a suitable device. In a next step 2 patient identification and other information is captured and stored with ultrasound image frames in one or more image formats including, e.g. DICOM. Next 3 the image is encrypted and transmitted by secured transmission to a Patient Data Storage (PDS) which in one embodiment is cloud-based. Then 4 the image is retrieved for processing including, without limitation, speckle reduction and contrast enhancement to improve image quality and image boundary information is preserved also. Next 5 to delineate free fluid or air, if any, morphological operation is carried out on the improved image and a desired threshold may be applied to trace the contour of the fluid or air accumulated area. Then, in the next step 6, free fluid or air accumulation is quantified in terms of percentages/pixels and the processed image is transmitted to PDS and saved along with the original image. Next 7, the processed image is accessed securely for review of the analysis and image, aiding in remote diagnosis of the patient by certified readers of ultrasound.

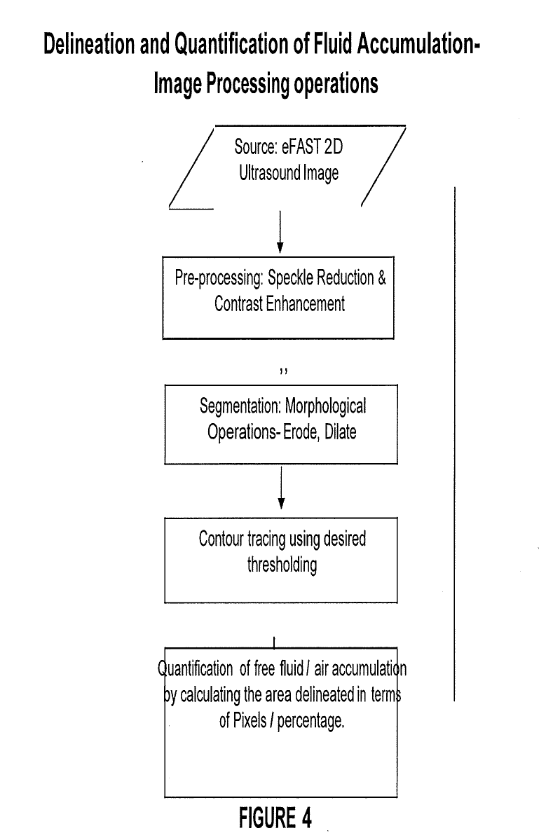

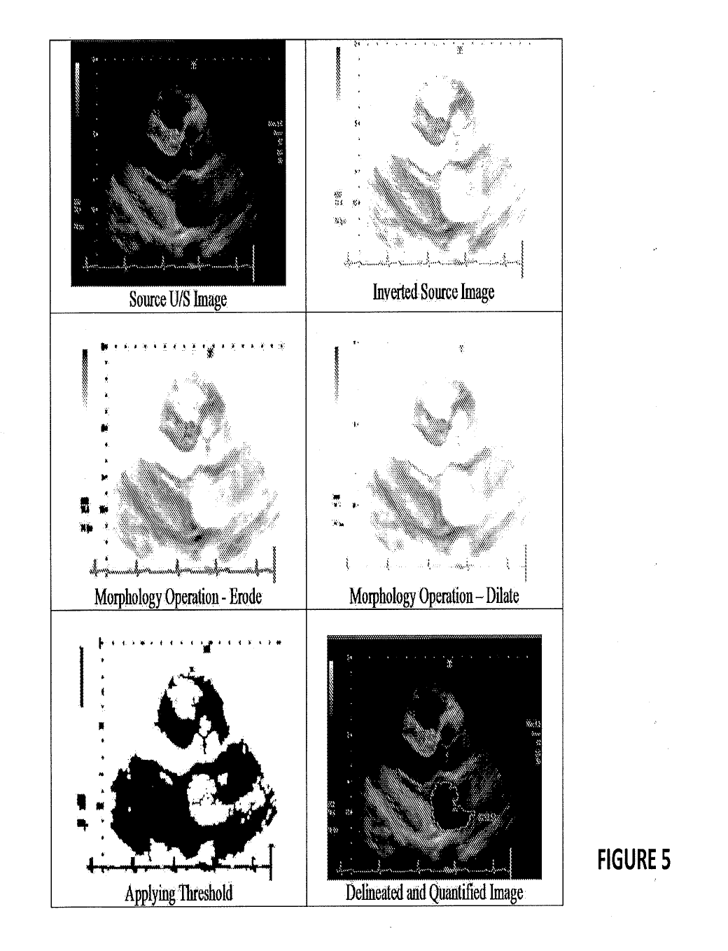

In the present invention, the eFAST Ultrasound Video is converted into a number of frames according to intervals of time. The delineation of free fluids or air or tissues of ultrasonic images is difficult due to the existence of noise and speckle. The pre-processing operations (shown in FIG. 4 and examples of the operations are shown in FIG. 5) in one embodiment include, without limitation, speckle reduction (de-speckle) using anisotropic diffusion filter. Other image enhancement techniques will be known to others with ordinary skill in the art. This process will improve the image quality significantly at the same time preserve the important boundary information, improving the image quality by contrast enhancement.

The invention, in one embodiment, employs segmentation to enhance image quality by increasing contrast and noise reduction by means of several steps. An image is segmented into background and object regions using morphology operations techniques for extracting image components which are useful in representation and description of region shape. Morphology operations include dilation, erosion, opening and closing. There is a structuring element (SE) which governs the operation. SE is a matrix which is applied (‘convolved’) to the matrix representation of the image. Dilation smoothes an image and bridges gaps (‘dilated’). Erosion removes irrelevant details. Opening smoothes the contour of an object, and removes thin protrusions while closing smoothes and fills gaps in the contour.

A threshold may be applied to an image to improve the object's border. An image histogram may be employed to select a threshold value between the maximum and minimum gray levels (for example between 0-255). The thresholding transformation (using the threshold value selected) improves the object border.

Using several different means, the invention can quantify a segmented area of an image and report the area for analyis. For example, an image in a DICOM image can be quantified by Pixel Spacing in millimeters or microns. In a non DICOM file the area of the object may only be calculated in Pixels and takes into account permissible limits (of area) to determine significance.

### Benefits

The utility of the exam using the invention is unparalleled, because it is rapid, performed at bedside, and may dictate whether the patient goes to the operating room or is observed. It is recommended as a primary modality of diagnostic testing in the primary sequence of the Advanced Trauma Life Support algorithm. Broadening the accessibility of ultrasound image analysis and transmission is critical to the reducing delay in patient disposition, thereby reducing mortality and morbidity in the “golden hour” of trauma. In trauma, mortality has one of three peaks—immediate, within the “golden hour”, and in weeks subsequent to the trauma, from infection or complications of surgery or hospital care. The eFAST exam itself addresses injuries that should be quickly identified early in the “golden hour.”

While the foregoing has been with reference to a particular embodiment of the invention, it will be appreciated by those skilled in the art that changes in this embodiment may be made without departing from the principles and spirit of the disclosure, the scope of which is defined by the appended claims.

1. A method for delineating and quantifying fluid and/or air accumulation in eFAST ultrasound images acquired from handheld/portable Ultrasound device by (1) automatically detecting the contour of fluid and/or air in accumulated area (2) calculating the area of fluid accumulation and reporting.

2. The method of claim 1, wherein said automatically detecting the contour of fluid accumulation using image processing techniques (Invert the image, Morphology-Dilate, Erode and applying the desired threshold and finally tracing the contour of the fluid accumulated area.

3. The method of claim 2, wherein said quantifies the amount of free fluid/air accumulation (if any) by calculating the delineated area in pixels/percentage.

4. A system for delineating and quantifying fluid and/or accumulation in eFAST ultrasound images acquired from handheld/portable Ultrasound device by (1) automatically detecting the contour of fluid and/or air in accumulated area (2) calculating the area of fluid accumulation and reporting.
